(19) United States
(12) Patent Application Publication
(10) Pub. No.: US 2008/0147100 A1
(43) Pub. Date: Jun. 19, 2008

(54) ANEURYSM NECK OBSTRUCTION DEVICE

(75) Inventor: Michael P. Wallace, Fremont, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED, INC., Maple Grove, MN (US)

(21) Appl. No.: 12/039,475

(22) Filed: Feb. 28, 2008

Related U.S. Application Data
(63) Continuation of application No. 10/374,520, filed on Feb. 25, 2003.
Application No. 10/374,520 is a continuation-in-part of application No. 09/886,567, filed on Jun. 21, 2001, now Pat. No. 6,454,780.
Application No. 09/886,567 is a continuation-in-part of application No. 10/219,930, filed on Aug. 15, 2002, now abandoned.

(51) Int. Cl. A61B 17/12

(52) U.S. Cl. 606/151

(57) ABSTRACT

An implantable medical device for at least partially closing a portion of a vascular aneurysm is disclosed. The treatment device includes a neck bridge having a delivery configuration and a deployed configuration. The device also includes an actuation mechanism configured to be temporarily engaged to the neck bridge to convert the neck bridge between the delivery configuration and the deployed configuration.

### RELATED APPLICATION DATA

The present application is a continuation of U.S. patent application Ser. No. 10/374,520, filed Feb. 25, 2003, which is a continuation-in-part of U.S. patent application Ser. No. 09/886,567, filed Jun. 21, 2001, now U.S. Pat. No. 6,454,780, and Ser. No. 10/219,930, filed Aug. 15, 2002, the content of which are incorporated herein by reference in their entirety.

### BACKGROUND OF THE INVENTION

The present invention deals with a medical treatment device. While conceivably the device could be utilized in the context of a variety of body spaces, the present description, for the sake of brevity, will be focused primarily on the treatment of vascular aneurysms. Accordingly, the present invention deals with an aneurysm treatment device for at least partially obstructing the neck portion of a vascular aneurysm.

A vascular aneurysm can be described as a localized stretching or distension of an artery due to a weakening of the vessel wall. The vascular distension itself is often referred to as an aneurysm sac and is typically related to a defect in the muscular coating of the artery and is probably developmental in origin. The entrance area that leads from the vessel to the aneurysm sack is often referred as an aneurysm neck. Often an aneurysm can be the site of internal bleeding and, catastrophically, the site of a stroke.

Several methods of treating aneurysms have been attempted, with varying degrees of success. At present, the treatment of aneurysms with drugs is substantially ineffective. Also, extra-vascular surgery, referred to as open craniotomy, for the purpose of preserving the parent artery is replete with disadvantages. A patient subject to open craniotomy for intercranial aneurysms typically must undergo general anesthesia, surgical removal of part of the skull, brain retraction, dissection around the neck of the aneurysm sac, and placement of a clip on the parent artery to prevent bleeding or rebleeding.

Alternative treatments include endovascular occlusion where the interior of the aneurysm is entered with a guidewire or a microcatheter. An occlusion is formed within the sac with an intention to preserve the parent artery. One means for forming the occlusion is through the introduction of an embolic agent within the sac. Examples of embolic agents include detachable coils, which are detached from the end of a guidewire, liquid polymers that polymerize rapidly on contact with blood to form a firm mass, and embolic particles delivered through a catheter.

Endovascular occlusion is not without drawbacks. For example, there is a risk of overfilling the sac and consequent embolic agent migration into the parent vessel. Overfilling of the sac can also generate undesirable additional pressure in the aneurysm.

Aneurysms that have a particularly wide opening between the aneurysm sac and the parent vessel (“wide neck aneurysm”) present difficulties concerning the retention of embolic materials. Specifically, wide neck aneurysms make it very difficult to maintain embolics (or other occlusive materials) within the aneurysm sac. This is especially true of liquid embolic materials. Of course, should the embolic material enter the parent vessel, it poses an undesirable risk of occlusion in the parent vessel.

Another means for forming a mass in an aneurysm sac involves the placement of an elastic expandable balloon in the aneurysm. Detachable occlusion balloons have been used for a number of medical procedures. These balloons are typically carried at the end of a catheter and, once inflated, are detached from the catheter. Such a balloon may be positioned within an aneurysm, filled and then detached from the catheter. Deploying the balloon within the aneurysm can be rather difficult due to the high rates of blood flow through the aneurysm.

In addition to delivery complications, elastic balloons have exhibited other problems with respect to performance in the context of vascular aneurysms. For example, as the balloon is inflated within an aneurysm, the operator must be very careful not to overfill the balloon due to possible risk of rupturing the aneurysm. Accordingly, following inflation, the balloon may be too small, potentially resulting in a release of the balloon from the aneurysm into the blood stream. Furthermore, the balloon often does not mold or shape to the odd-shaped contours of the aneurysm, leaving room for blood to continue flowing through the aneurysm, or generating undesired pressure on the aneurysm walls.

Another means for treating vascular aneurysms involves the placement of a liner in the aneurysm sac. An aneurysm liner includes a liner sac that is placed in the aneurysm sac and filled so as to occlude the aneurysm. A guidewire is typically utilized to carry the liner through the vasculature and to assist in deploying the liner in the aneurysm.

While the aneurysm liner concept is intuitively attractive, it has posed a number of technical challenges. One primary challenge involves the difficulty in producing a material that is robust enough to contain embolic material without inhibiting the ability of the embolics to conform to the aneurysm geometry itself, rather than the geometry of the liner. In many instances, materials currently incorporated into aneurysm liner concepts are not compliant enough to adequately remodel the neck portion of an aneurysm sac. This disadvantage can lead to neck remnants and subsequently recanalization after embolization.

Most current aneurysm liners are physically inconvenient or inappropriate for treatment of large aneurysms. For example, many liner concepts involve forming the aneurysm liner of a woven or braided polymeric material such as polypropylene or polyester. These mesh materials are difficult to use in treating medium to large size aneurysms, for example, aneurysms 5-20 millimeters in diameter. Such mesh materials result in an assembly that is too bulky when collapsed down into the catheter for delivery. In other words, the amount of liner material required to fill a relatively large aneurysm is very difficult to collapse down into a constrained, low profile, delivery configuration small enough to be delivered and deployed without excess friction on the walls of the delivery catheter or other delivery lumen. The bulkiness of these devices makes them inconvenient or inappropriate for intra-cranial delivery.

It should also be noted that many current aneurysm liner concepts lack consistent and effective expansion systems or concepts. A consistent and effective expansion reduces procedural complications associated with transformation of liners from a constrained state in a delivery catheter to an unconstrained state when deployed in an aneurysm.

### SUMMARY OF THE INVENTION

The present invention is an aneurysm treatment device for treating aneurysms of various shapes and sizes.

One embodiment pertains to an implantable medical device for at least partially closing a portion of a vascular aneurysm. The treatment device includes a neck bridge having a delivery configuration and a deployed configuration. The device also includes an actuation mechanism configured to be temporarily engaged to the neck bridge to convert the neck bridge between the delivery configuration and the deployed configuration.

Another embodiment pertains to a method of treating an aneurysm in a parent vessel having a lumen, the aneurysm having a neck and inner wall defining a cavity that is in communication with the lumen. The method includes providing a collapsible neck bridge configured to be temporarily engaged by an actuation mechanism. The collapsible neck bridge is endovascularly moved in a delivery configuration, wherein the actuation mechanism is engaged to the collapsible neck bridge, to a site proximate the aneurysm. The actuation mechanism is disengaged from the collapsible neck bridge so as to covert the collapsible neck bridge from the delivery configuration to a deployed configuration.

Another embodiment pertains to a system for treating an aneurysm in a vessel, the aneurysm having an inner wall and a neck defining a cavity. The system includes a treatment device and an actuation mechanism. The treatment device includes a collapsible neck bridge configured to be temporarily engaged by the actuation mechanism. The collapsible neck bridge is in a delivery configuration when the actuation mechanism is engaged thereto, and in a deployed configuration when the actuation mechanism is disengaged there from.

### BRIEF DESCRIPTION OF THE DRAWINGS

FIGS. 1A and 1B are partially broken away side views of an aneurysm treatment device having a collapsible neck bridge, wherein FIG. 1A shows the collapsible neck bridge in a delivery configuration and FIG. 1B shows the collapsible neck bridge in a deployed configuration.

FIGS. 2A-2C are partially broken away side views that illustrate deployment of the aneurysm treatment device of FIGS. 1A and 1B within a vascular aneurysm.

FIG. 2D is a partially broken away side view that illustrates delivery of an embolic agent through the aneurysm treatment device of FIGS. 1A and 1B.

FIG. 2E is a partially broken away side view that illustrates detachment of the collapsible neck bridge of FIGS. 1A and 1B from an elongated delivery member.

FIG. 3 is a partially broken away side view of an aneurysm treatment device that includes a sheath for encouraging an effective delivery of an embolic agent into an aneurysm.

FIG. 4A is an accordion-shaped shape memory actuator having an elongated form.

FIG. 4B is the accordion-shaped shape memory actuator of FIG. 4A but in a constricted form.

FIG. 5A is a partially broken away side view of an aneurysm treatment device that incorporates the accordion-shaped shape memory actuator of FIGS. 4A and 4B, the aneurysm treatment device having a collapsible neck bridge that is depicted in a delivery configuration.

FIG. 5B is a partially broken away side view that illustrates deployment of the aneurysm treatment device of FIG. SA within a vascular aneurysm.

FIG. 6A is a partially broken away side view of an aneurysm treatment device that incorporates a shape memory strut actuator, the aneurysm treatment device having a collapsible neck bridge that is depicted in the delivery configuration.

FIG. 6B is a partially broken away side view of the aneurysm treatment device of FIG. 6A, but with the collapsible neck bridge depicted in the deployed configuration FIG. 7A is a perspective side view of a collapsible aneurysm obstruction device having a deployed configuration.

FIG. 7B is a side view of the device of FIG. 7A but in a delivery configuration.

FIG. 7C is a side view of the device of FIG. 7B but further incorporating the accordion-shaped shape memory actuator of FIGS. 4A and 4B.

FIG. 7D is a partially broken away side view that illustrates deployment of the aneurysm treatment device of FIG. 7C within a vascular aneurysm.

FIG. 8A is a partially broken away side view of the aneurysm treatment device having a neck bridge in a delivery configuration.

FIG. 8B is a partially broken away side view of the aneurysm treatment device of FIG. 8A having the neck bridge in a deployed configuration.

FIG. 8C is a partially broken away side view of the aneurysm treatment device of FIG. 8A wherein an actuation mechanism is released from the deployed neck bridge.

FIG. 8D is a top view of the aneurysm treatment device of FIG. 8A.

FIG. 9A is a partially broken away side view of the aneurysm treatment device having a neck bridge in a delivery configuration.

FIG. 9B is a partially broken away side view of the aneurysm treatment device of FIG. 9A having the neck bridge in a deployed configuration.

FIG. 9C is a partially broken away side view of the aneurysm treatment device of FIG. 9A wherein an actuation mechanism is released from the deployed neck bridge.

FIG. 9D is a top view of the aneurysm treatment device of FIG. 9A.

### DETAILED DESCRIPTION OF THE ILLUSTRATIVE EMBODIMENTS

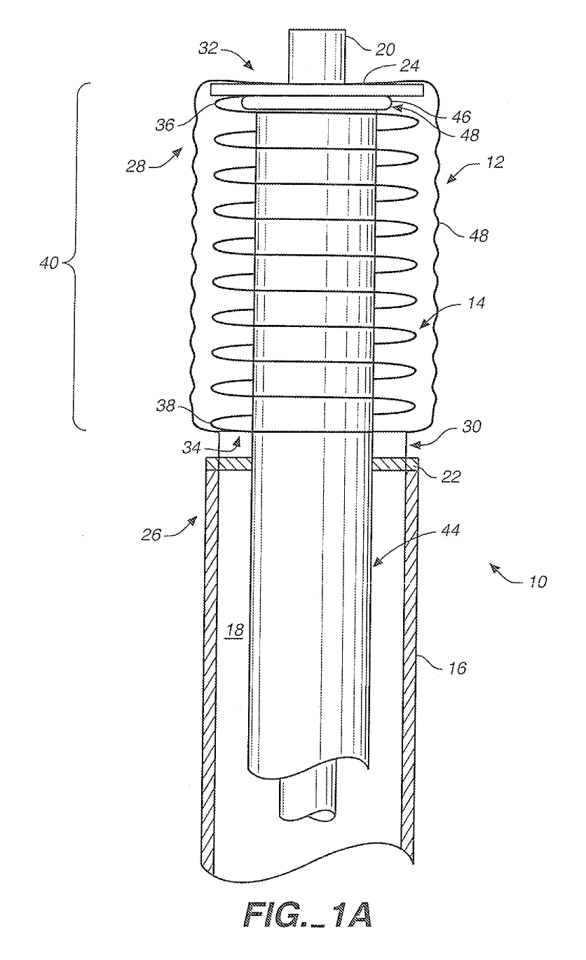

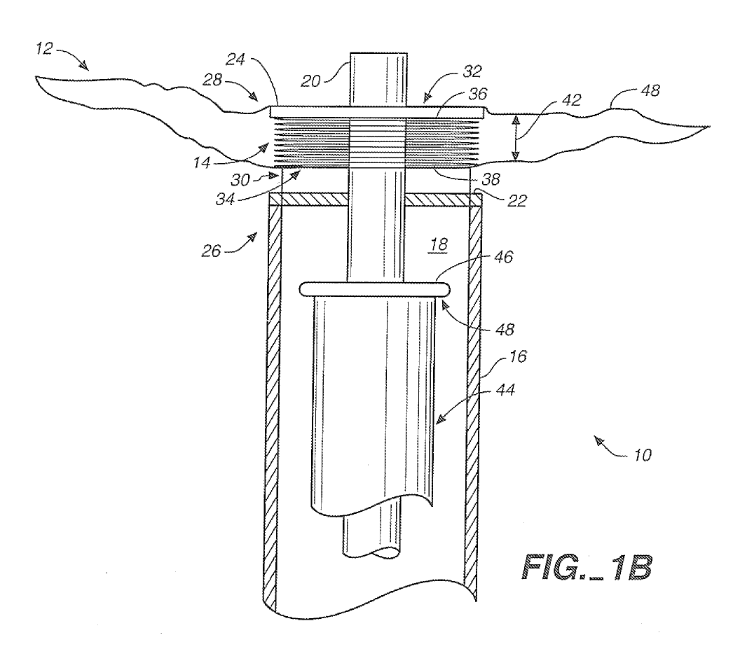

FIGS. 1A and 1B are partially broken away side views of an aneurysm treatment device 10 in accordance with an embodiment of the present invention. FIG. 1A shows treatment device 10 with a neck bridge 12 in a delivery configuration, while FIG. 1B shows device 10 with collapsible neck bridge 12 in a deployed configuration. The same reference numerals used in FIG. 1A are also used in FIG. 1B for elements that are the same or similar in both drawings.

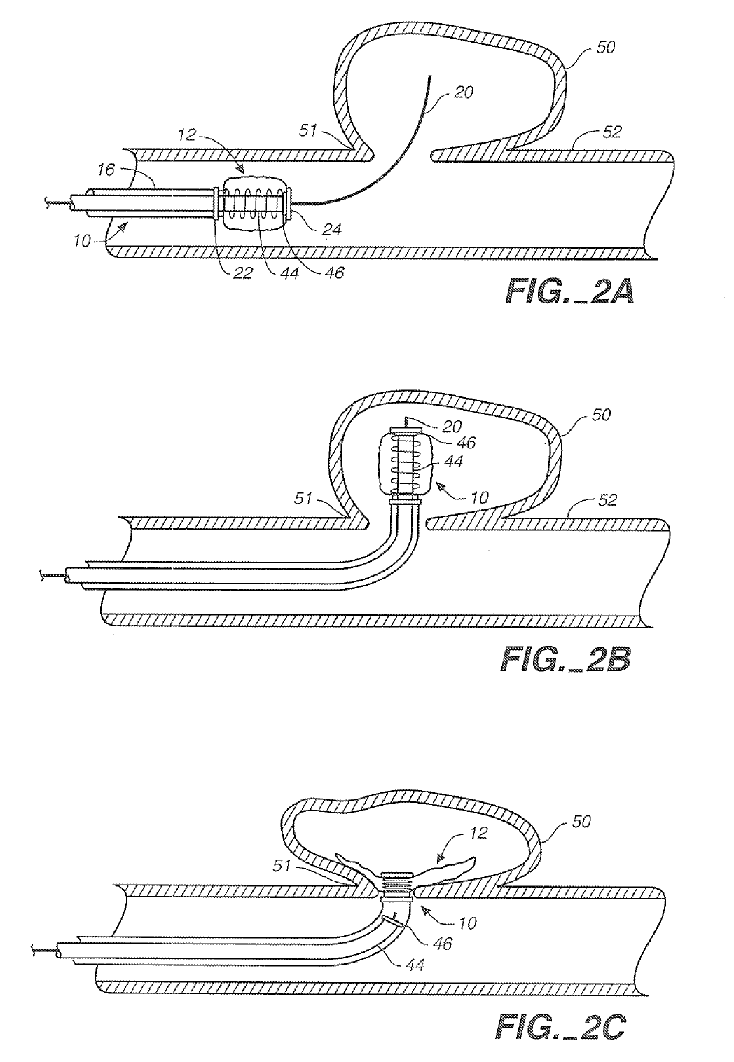

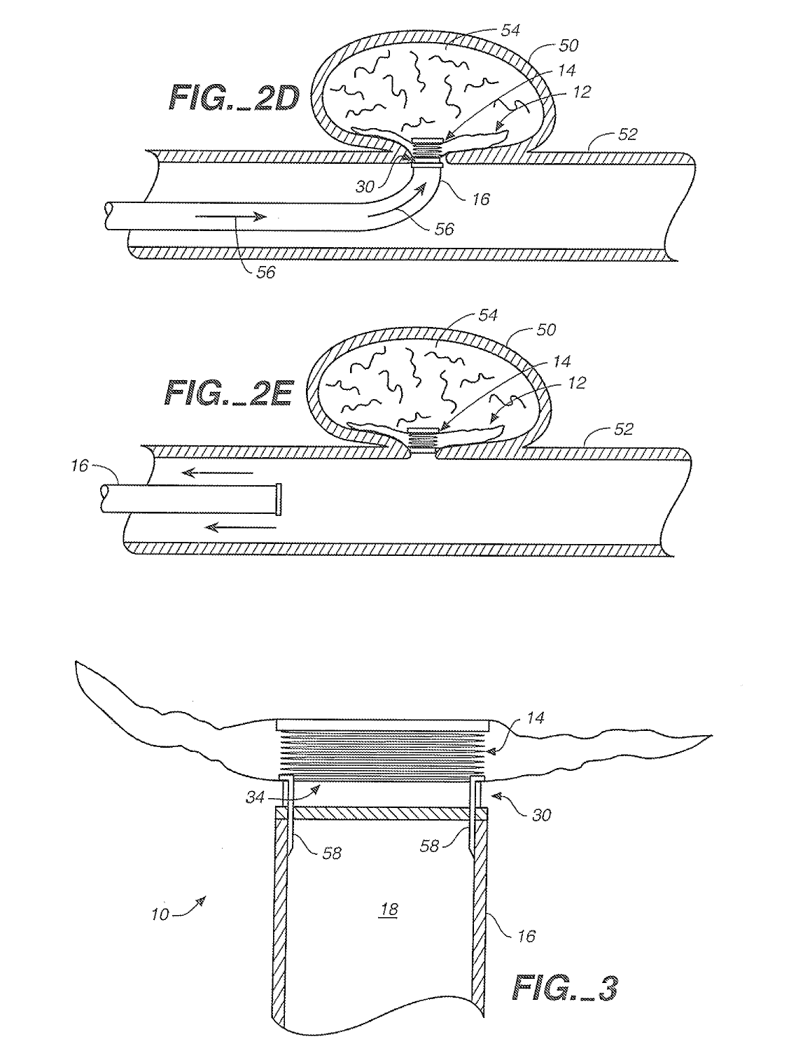

The FIG. 1A delivery configuration is illustratively designed to facilitate smooth and efficient intravascular delivery of treatment device 10 to an internal location proximate an aneurysm. In the FIG. 1A delivery configuration, collapsible neck bridge 12 is of a size and overall flexibility to accommodate effective and efficient intravascular delivery to an aneurysm site. Also, when neck bridge 12 is in the delivery configuration, it is of a size and overall flexibility to be deliverable through a tubular delivery device, such as through a delivery catheter (not illustrated). Delivery and subsequent deployment of device 10 will be described in greater detail in relation to FIGS. 2A-2C.

Aneurysm treatment device 10 illustratively includes an actuation mechanism 14 that is operably attached to collapsible neck bridge 12 and is configured to convert collapsible neck bridge 12 between the delivery configuration of FIG. 1A and the deployed configuration of FIG. 1B. When collapsible neck bridge 12 is in the FIG. 1A delivery configuration, actuation mechanism 14 illustratively has the depicted elongated form. When collapsible neck bridge 12 is in the FIG. 1B deployed configuration, actuation mechanism 14 illustratively has the depicted constricted form.

In accordance with an embodiment of the present invention, neck bridge 12 and attached actuation mechanism 14 are implantable within an aneurysm. To accommodate intravascular delivery of these implantable elements, an elongated delivery member 16 is detachably connected to actuation mechanism 14. Alternatively, the detachable connection could be made to collapsible neck bridge 12. Elongated delivery member 16 is illustratively a microcatheter and includes a hollow portion 18 through which different items or materials, such as embolic agents or a guide wire 20, can be transferred or delivered. Guide wire 20 is a known element for use in intravascular navigation and can optionally be incorporated into the present invention to facilitate delivery of device 10 to an internal site proximate an aneurysm. Guide wire 20 illustratively extends through a lumen that is continuous through delivery member 16, joint 30 (described in more detail below), through apertures in neck bridge 12 (described in more detail below), and through actuation mechanism 14.

Radio opaque band or marker 22 located on a distal end 26 of elongated delivery member 16, as is known in the art, could optionally be incorporated into device 10 to assist in the guidance of elongated delivery member 16 through a vascular system utilizing principles radiography or fluoroscopy. Alternatively or additionally, a different radio opaque band or marker 24 could illustratively be included on a distal end 28 of collapsible neck bridge 12 and could be utilized for similar internal navigation purposes.

In accordance with one embodiment, attachment between elongated delivery member 16 and actuation mechanism 14 (or between member 16 and collapsible neck bridge 12) is of a detachable nature. Illustratively, aneurysm treatment device 10 includes a severable joint 30 to facilitate detachment. Severable joint 30 includes means for severing elongated delivery member 16 from collapsible neck bridge 12 and actuation mechanism 14. The severing action of joint 30 illustratively enables collapsible neck bridge 12 and actuation mechanism 14 to remain implanted within an aneurysm (illustratively in the FIG. 1B deployed position) after most or all other elements, including guidewire 20 and elongated delivery member 16, have been removed from the associated vascular system. In accordance with one illustrative embodiment, severable joint 30 causes severance via mechanical means or via a known electrolytic dissolution of a sacrificial joint. Means other than mechanical or electrolytic, however, should be considered within the scope of the present invention. For the purpose of simplifying description, it will be assumed that severable joint 30 is an electrolytically severable joint. It should be noted that the Figures reflect this embodiment of the present invention. Illustratively, joint 30 alone is constructed of a material susceptible to electrolytic dissolution in blood and dissolves in response to an intentionally timed and applied electrolytic signal.

In accordance with the embodiments of FIGS. 1A and 1B, actuation mechanism 14 is a resilient member operably disposed to bias collapsible neck bridge 12 toward the. deployed configuration of FIG. 1B. In the FIG. 1A and FIG. 1B embodiments, actuation mechanism 14 is illustrated as being a spring. Illustratively, the spring is constructed of a flexible metallic or plastic material. Other similar resilient members including but not limited to other springs, flexible or elastic straps, or other resilient components (i.e., structures constructed of sponge-like material, rubber, a flexible polymeric material, a plastic material, etc.) should be considered within the scope of the present invention. In accordance with one embodiment, a material having shape memory characteristics could be utilized to form an appropriate resilient member.

In accordance with the FIG. 1A and FIG. 1B embodiments of the present invention, actuation mechanism 14 is located inside of collapsible neck bridge 12 and defines a lumen or cylindrical hollow column therethrough. Collapsible neck bridge 12 illustratively includes a distal aperture 32 and a proximal aperture 34 formed therein. The lumen defined within actuation mechanism 14 illustratively is substantially aligned in the same axis as distal aperture 32 and proximal aperture 34. Actuation mechanism 14 includes a first end 36 connected proximate to distal aperture 32, and a second end 38 connected proximate to proximal aperture 34. Illustratively, when collapsible neck bridge 12 is in the FIG. 1A delivery configuration, distal aperture 32 and proximal aperture 34 are spaced apart a first distance 40. When collapsible neck bridge 12 is in the FIG. 1B deployed configuration, distal aperture 32 and proximal aperture 34 are spaced apart a second distance 42 that is less than first distance 40.

In accordance with one embodiment of the present invention, guide wire 20 is a first elongate member that slidably extends through hollow portion 18 of elongated delivery member 16, through an opening formed in distal end 26 of delivery member 16, through a hollow joint 30, through proximal aperture 34 in neck bridge 12, through the lumen formed in actuation mechanism 14 and out distal aperture 32 in neck bridge 12. Optional markers 22 and 24 are configured so as to not interfere with such an extension of guide wire 20. Illustratively, an operator of device 10 has control of guide wire 20 from a proximal end thereof and can extend or withdraw guide wire 20 as desired.

In accordance with another embodiment of the present invention, a second elongate member 44 (second in relation to guide wire 20, which is the first elongate member) has a ball valve 46 connected to a distal end 48 thereof. The second elongate member 44 and ball valve 46 coaxially and slidably engage guide wire 20. Illustratively, second elongate member 44 and ball valve 46 are extendable through device 10, similar to guide wire 20, except that ball valve 46 is sized so as to engage an area inside neck bridge 12 proximate aperture 32. For example, ball valve 46 might engage a material terminus formed by neck bridge 12 material, an intentionally sized and positioned metal ring (not illustrated), or marker 24. Regardless of the precise structural arrangement, conceptually, ball valve 46 and second elongate member 44 are illustratively not permitted to extend through distal aperture 32.

Accordingly, because actuation mechanism 14 (i.e., a resilient member) is configured to bias neck bridge 12 toward the FIG. 1B deployed configuration, an operator having control (i.e., from a proximal end of elongated member 44) can convert device 10 between the FIG. 1A delivery configuration and the FIG. 1B deployed configuration. To do so, the operator need simply vary the amount of pressure applied by ball valve 46 against the area proximate distal aperture 32. This system of control is apparent in FIG. 1A and FIG. 1B in that in FIG. 1A, ball valve 46 is illustratively engaging an area inside neck bridge 12 proximate distal aperture 32 and is illustratively being supplied with enough pressure to overcome the bias of actuation mechanism 14 and place neck bridge 12 and device 10 in the illustrated delivery configuration. Conversely, in FIG. 1B, ball valve 46 has been withdrawn from actuation mechanism 14, thereby enabling conversion to the illustrated deployed configuration. It should be noted that guide wire 20 is an optional element and that second elongate member 44 could perform the conversion function without existence or assistance from guide wire 20.

It should be pointed out that when neck bridge 12 and device 10 are in the FIG. 1B deployed configuration, neck bridge 12 is illustratively approximately disc-shaped and of a size and overall flexibility to be lodged at the neck portion of an aneurysm (described in more detail in relation to FIGS. 2A-2E). In accordance with one embodiment, neck bridge 12 is intentionally designed to have a diameter significantly greater than the diameter of a targeted aneurysm neck opening. In accordance with one embodiment, as is illustrated by. FIG. 1B, a deployed neck bridge 12 can take the form of a double-layered aneurysm occlusion member. Accordingly, because the occlusion member is double-layered, the material incorporated into neck bridge 12, illustratively material 48 can be light weight or at least less bulky than would be necessary for a single layer aneurysm obstruction device. The present design enables a light weight material to be streamlined during delivery and yet still form a solid, potentially two-layered, occlusion in an implanted delivery configuration. In accordance with one embodiment, material 48 is permeable to blood flow. In accordance with other embodiments, material 48 is a mesh material or a braided polymeric or metal material.

In accordance with one embodiment, material 48 is a bio-compatible fabric-like material, such as a braided or woven polymeric material. Illustratively, whether in a fabric-like form or not, material 48 may be constructed with polymers that include but are not limited to polyethylene, polypropylene, polyvinylchloride, polyamides such as Nylon, polyurethanes, polyvinylpyrrolidone, polyvinyl alcohols, polyvinylacetate, cellulose acetate, polystyrene, polytetrafluoroethylene- , polyesters such as polyethylene terphthalate (Dacron), silk, cotton, and the like. In accordance with one embodiment, material 48 can be constructed with biodegradable material, such as (but not limited to) polylatic acid or polyglycolic acid. The above listed material are only examples of the broad range of materials that could be incorporated into material 48. In accordance with one embodiment, a radio-opaque material is woven or otherwise incorporated into material 48 (i.e., tantulum platinum, gold, etc.) to facilitate and assist in guidance through a vascular system utilizing principles of radiography or fluoroscopy.

FIGS. 2A-2C are partially broken away side views that illustrate deployment of the aneurysm treatment device 10 within a vascular aneurysm 50. The same reference numerals used in FIGS. 2A-2C for elements that are the same or similar to elements previously described in relation to other Figures. Also, numerals used to identify newly described elements in FIG. 2A are similarly numbered and represent the same or similar elements in FIGS. 2B and 2C, and subsequently in FIGS. 2D and 2E.

FIG. 2A illustrates a partially sectioned view of an aneurysm 50 emanating from the wall of a feeding vessel 52. Illustratively, guide wire 20 extends through a neck portion 51 of aneurysm 50. Guide wire 20 can be utilized to assist in the delivery of aneurysm treatment device to a site within aneurysm 50. In accordance with one embodiment, guide wire 20 is placed in the vasculature first. Once the distal end of guide wire 20 is moved past the aneurysm neck 51, elongated delivery member 16 is advanced over guide wire 20 until aneurysm treatment device 10 is in place within aneurysm 50. Other methods of intravascular delivery, with and without incorporation of a guide wire, are known in the art and should be considered within the scope of the present invention. Markers 22 and 24, as is known in the art, are illustratively radio-opaque bands or markers that can be utilized to assist in the guidance of device 10 through a vascular system utilizing principles of radiography or fluoroscopy.

In FIG. 2A, in accordance with an embodiment of the present invention, second elongate member 44 and ball valve 46 are operated as described above to maintain the collapsible neck bridge 12 in a streamlined, low profile, delivery configuration during insertion and manipulation of device 10 within aneurysm 50.

FIG. 2B shows that treatment device 10 has been positioned through vessel 52 and neck 51 into a sac portion of aneurysm 50. At this point, second elongate member 44 and ball valve 46 can be withdrawn, as described above, to covert neck bridge 12 to a deployed configuration. Guide wire 20, if being utilized, can also be withdrawn.

FIG. 2C shows that treatment device 10 has been converted to a deployed configuration following withdrawal of second elongated member 44 and ball valve 46. As is illustrated, neck bridge 12, in the deployed configuration, obstructs (or at least partially closes) neck 51 of aneurysm 50. It should be noted that while aneurysm 50 is generally illustrated as a symmetrically shaped aneurysm, asymmetrically shaped aneurysms having a variety of neck 51 shapes could be treated utilizing the teachings of the present invention.

Referring to FIG. 1B, in accordance with an embodiment of the present invention, when collapsible neck bridge 12 is in the deployed configuration and actuation mechanism 14 is therefore constricted, actuation mechanism 14 forms a conduit through which an embolic agent or other aneurysm treatment device can be delivered to a location within an aneurysm. As described above in relation to guide wire 20, there is illustratively a continuous hollow or open chamber or lumen through elongated delivery member 16, joint 30 and neck bridge 12. This chamber, in combination with the conduit formed by the collapsed actuation mechanism 14 illustratively creates an effective and appropriate path for an operator of device 10 to deliver materials through elongated delivery member 16 and directly into an aneurysm. This direct delivery of an embolic or other treatment device, combined with containment of the treatment agent by neck bridge 12, is of particular benefit because the treatment device delivered into the aneurysm is allowed to closely conform to the actual interior of the aneurysm, rather than having to conform to the aneurysm through a bulky aneurysm liner.

In accordance with another embodiment, with further reference to FIG. 1B, embolic or other aneurysm treatment devices can be alternatively delivered to an interior portion of an aneurysm. Illustratively, ball valve 46 can be brought into adjacent contact with aperture 32 in a constricted neck bridge 12. Embolic or other material can then illustratively be delivered through a lumen, formed within second elongate member 44, to a location within the interior of an aneurysm. Illustratively, guide wire 20 could be removed from member 44 to facilitate this type of material delivery.

FIG. 2D is a partially broken away side view that illustrates an embodiment of the present invention, wherein an embolic agent or another aneurysm treatment device can be delivered to a location within aneurysm 50. Similar elements are similarly numbered in FIG. 2D to reflect elements described in relation to the previous Figures. Illustratively, a plurality of occlusion coils 54 have been delivered, along the path demonstrated by arrows 56, through elongated delivery member 16, through joint 30, through the conduit formed by retracted actuation mechanism 14, through the above described apertures in neck bridge 12 and into aneurysm 50. Deliverable embolic and aneurysm treatment devices other than occlusive coils, of course, are within the scope of the present invention and can be similarly delivered. Other deliverable agents include but are not limited to coils of many shapes and sizes, particles, liquids, and supporting members. Illustratively, joint 30 can be severed as described above to leave actuation mechanism 14 and neck bridge 12 in a secure implanted state. Illustratively, as described above, an electrolytic signal could be transferred through the blood in vessel 52 so as to dissolve joint 30.

FIG. 2E is a partially broken away side view that illustrates actuation mechanism 14 and neck bridge 12 following implantation and following removal of non-implanted elements from the vascular system. Similar elements are similarly numbered in FIG. 2D to reflect elements described in relation to the previous Figures. Illustratively, the plurality of occlusion coils 54 are contained within aneurysm 50 by the implanted elements of aneurysm treatment device 10. Delivery member 16 is illustratively removed from vessel 52.

FIG. 3 is a partially broken away side view of an aneurysm treatment device 10 that includes a sheath portion 58. The same reference numerals are used in FIG. 3 for elements that are the same or similar to those elements described in relation to the previous Figures. Illustratively, the primary purpose of sheath 58 is to facilitate delivery of liquid embolic agents, or other deliverable aneurysm treatments, that might disadvantageously leak from or escape device 10 during delivery. In particular, there is a risk that such agents might escape from device 10 through areas proximate joint 30. To discourage leaks in that area, in accordance with an embodiment of the present invention, sheath 58 bridges between hollow portion 18 of elongated delivery member 16 and the conduit formed by activation mechanism 14 in the constricted configuration. In accordance with one embodiment, sheath 58 is removed with delivery member 16 following detachment of joint 30. In accordance with another embodiment, sheath 58 stays with activation mechanism 14 following detachment and is configured to collapse and seal proximal aperture 34 in neck bridge 12.

In accordance with an embodiment of the present invention, the above described actuation mechanism 14, rather than being a resilient member that physically biases neck bridge 12 and treatment device 10 toward a deployed configuration, is an actuation mechanism constructed of a shape memory polymer (SMP) material.

As will be described below, replacing a physically biasing actuation mechanism 14 with an actuation mechanism constructed of a SMP material enables conversion of a treatment device 10, such as the above-described conversion between the elongated and constricted forms of neck bridge 1-2, to be temperature-based rather than mechanically-based. Switching to temperature-based actuation effectively enables the elimination of second elongated member 44 and ball valve 46 (FIGS. 1A and 1B) from the above-described embodiments.

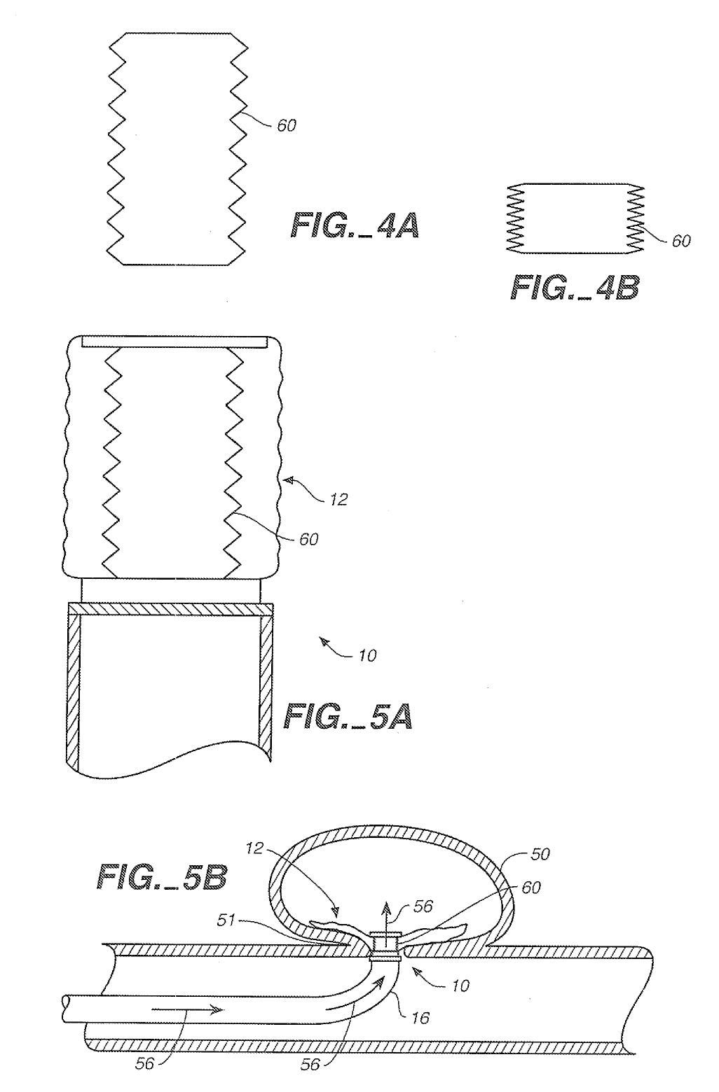

In accordance with an embodiment of the present invention, FIGS. 4A and 4B are side views of an accordion-shaped shape memory actuator 60 constructed of SMP material, wherein FIG. 4A shows actuator 60 having an elongated form and FIG. 4B shows actuator 60 having a constricted form. The FIG. 4A elongated form of actuator 60 illustratively represents the form of actuator 60 when at a temperature below a predetermined transition temperature. The FIG. 4B constricted form of actuator 60 illustratively represents the form of actuator 60 after the temperature of actuator 60 has been raised above the transition temperature. In accordance with one embodiment, after actuator 60 has converted into the FIG. 4B constricted form, actuator 60 remains in the constricted form regardless of subsequent temperature changes. In accordance with known methods, actuator 60 can originally be formed in the FIG. 4B constricted form via injection mold or clip processes in order to facilitate subsequent transformation back to the FIG. 4B constricted form from a FIG. 4A elongated form. It should be noted that the accordion shape of actuator 60 is only illustrative of the many potential shapes that could be utilized in forming SMP material into an actuator 60 configuration. In accordance with one embodiment, actuator 60 has a hollow core or lumen completely extending through a center axis thereof. In accordance with one embodiment, actuator 60, except for the described different means of actuation, is configured to operate within device 10 in a manner similar or identical to the operation of actuation mechanism 14 described above.

FIG. 5A is a partially broken away side view of an aneurysm treatment device 10 that incorporates the accordion-shaped SMP material actuator 60 of FIGS. 4A and 4B. The same reference numerals are used in FIG. 5A for elements that are the same or similar to those elements described in relation to the previous Figures.

Treatment device 10, in FIG. 5A, is substantially the same as described above but incorporates accordion-shaped, temperature-based actuator 60, rather than a mechanical resilient member. Accordingly, actuator 60 can convert from the illustrated elongated form to a constricted form, and neck bridge 12 can convert from the illustrated delivery configuration to a deployed configuration, without a mechanical pushing device, such as previously described second elongated member 44 (FIGS. 1A and 1B). When device 10 is within a vascular system, in accordance with an embodiment of the present invention, conversion of actuator 60 from the illustrated elongated form to a constricted form, and conversion of neck bridge 12 from the illustrated delivery configuration to a deployed configuration, can be accomplished by raising the temperature of actuator 60 above a transition temperature. Illustratively, although it could be otherwise accomplished, the temperature raise could be accomplished by transferring a warm bolus of saline to an internal environment proximate device 10 and actuator 60. The precise value of the transition temperature is dependent upon the particular incorporated SMP material and can illustratively be desirably selected by desirably selecting an SMP material.

FIG. 5B is a partially broken away side view of the treatment device 10 of FIG. 5A and illustrates deployment within aneurysm 50. The same reference numerals are used in FIG. 5A for elements that are the same or similar to those elements described in relation to the previous Figures.

In FIG. 5B, the temperature of actuator 60 has illustratively been raised above the transition temperature and neck bridge 12 has illustratively taken a deployed configuration so as to at least partially obstruct neck 51 of aneurysm 50. Illustratively, an embolic agent or other treatment devices could be delivered through elongated delivery member 16 and through the components of treatment device 10, including actuator 60, along the path of lines 56 and into aneurysm 50. Illustratively, joint 30 can be severed as described above to leave actuation mechanism 14 and neck bridge 12 in an implanted state, following removal of delivery member 16 from the associated vascular system.

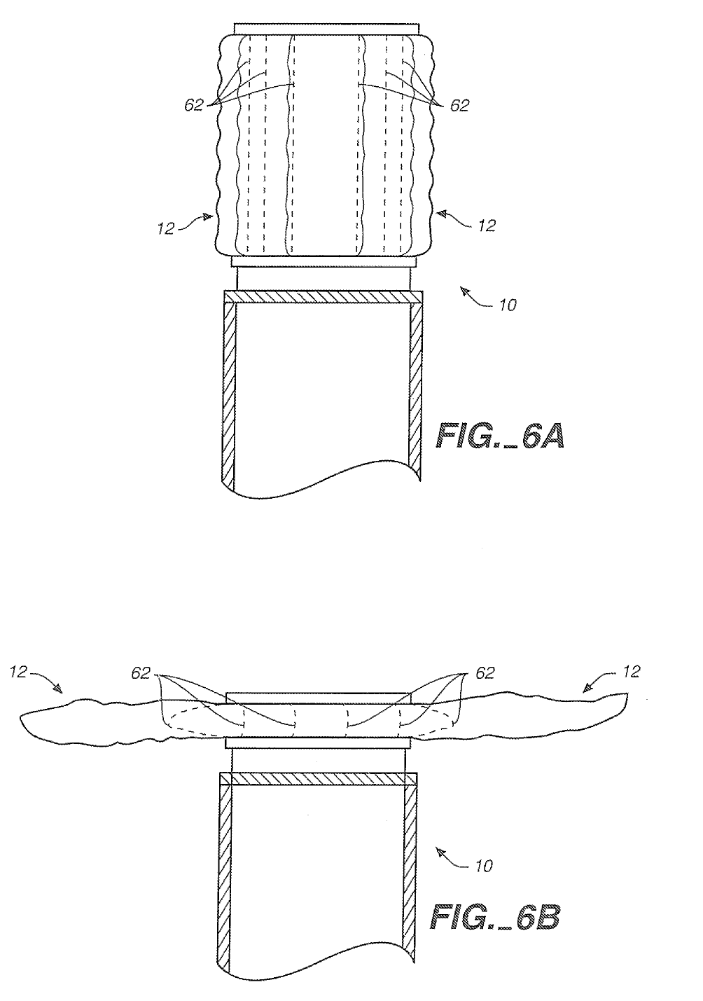

FIGS. 6A and 6B are partially broken away side views of embodiments of the present invention, wherein aneurysm treatment device 10 incorporates a SMP material-based actuator having a plurality of SMP material struts 62. The same reference numerals are used in FIGS. 6A and 6B for elements that are the same or similar to those elements described in relation to the previous Figures.

FIG. 6A illustrates collapsible neck bridge 12 in a delivery configuration and SMP material struts 62 in an elongated form, before the temperature of material struts 62 has been raised above a transition temperature. FIG. 6B illustrates collapsible neck bridge 12 in a deployed configuration and SMP material struts 62 in a constricted form, after the temperature of material struts 62 has been raised above a transition temperature. Illustratively, conversion from the FIG. 6A delivery configuration to the FIG. 6B deployed configuration takes place within an aneurysm. It should be noted that similar to previously described embodiments, device 10 in the FIG. 6B deployed configuration includes a continuous lumen or path through which embolic or other aneurysm treatment agents can be transferred into an aneurysm. Illustratively, constricted struts 62 form a path for delivery through neck bridge 12.

In accordance with one embodiment, the treatment device of FIGS. 6A and 6B operate in substantially the same manner as the treatment device of FIGS. 5A and 5B, except that the FIG. 5 embodiment incorporates accordion-shaped SMP material actuator 60 and the FIG. 6 embodiment incorporates strut-shaped SMP material actuator struts 62. The primary difference is illustratively in the shape and configuration of the incorporated shape memory actuator. In accordance with one embodiment, embolic agents or other treatment devices can be delivered through both the FIG. 5 and FIG. 6 embodiments. The construction of the incorporated shape memory actuator illustratively accommodates such delivery through the respective device 10 in both embodiments.

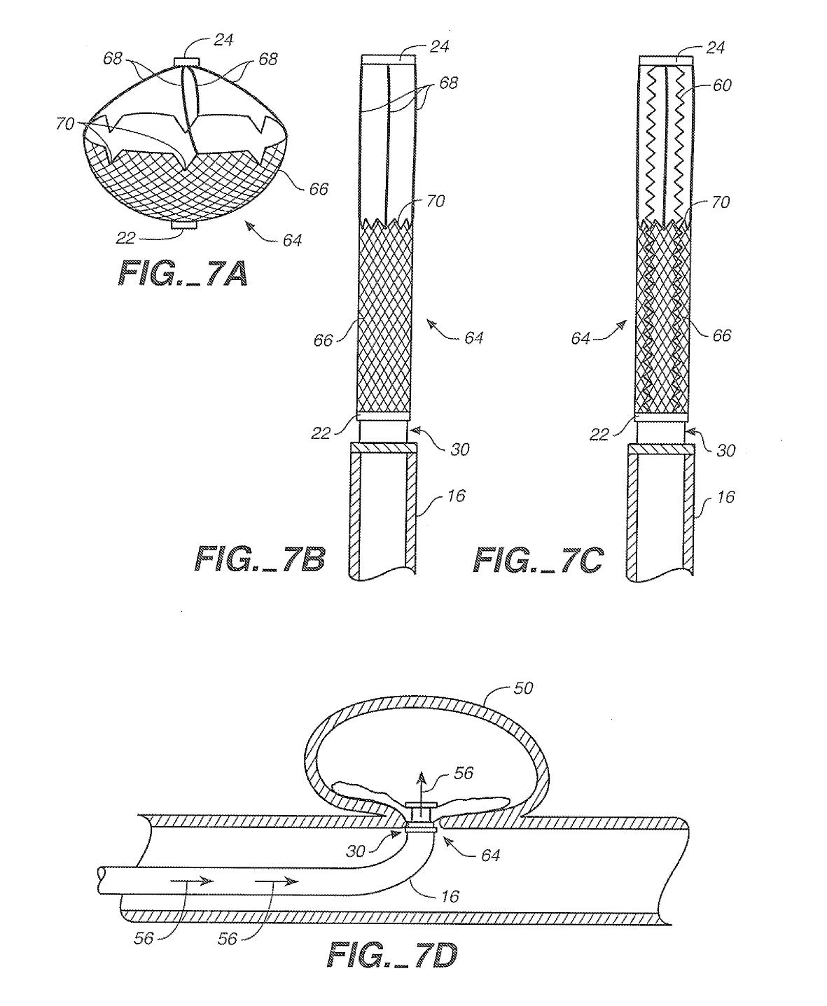

FIG. 7A is a perspective side view of a collapsible aneurysm obstruction device 64 having an inverted parachute configuration. The same reference numerals are used in FIG. 7A, as well as in subsequent FIGS. 7B-7D, for elements that are the same or similar to those elements described in relation to the previous Figures.

Referring to FIG. 7A, a single example of the many aneurysm treatment devices within which an actuating mechanism of the present invention could be incorporated is illustrated. Device 64 includes radio-opaque bands or markers 22 and 24 that operate as described above. While both marker 22 and 24 are optional elements, marker 22 illustratively serves as a connection point for a plurality of struts 68 that extend from a material base 66. In accordance with one embodiment, a connection point having non-marker characteristics could be substituted for marker 22. Struts 68 are illustratively connecting tethers. In accordance with one embodiment, struts 68 are constructed of shape memory material. Illustratively, struts 68 are utilized rather than additional material 66 so as to eliminate some of the bulk of device 64. In accordance with one embodiment, a portion of each strut 68 is woven into material 66. Material cut-outs 70 are an optional element of the present invention and illustratively enable a further elimination of bulk from the treatment device 64. Material 68 could illustratively be constructed of any of a number of materials suitable to obstruct the neck portion of an aneurysm. In accordance with one embodiment, the material utilized is permeable to blood flow.

FIG. 7B is a side view of device 64 after it has been collapsed into a delivery configuration and attached to an elongated delivery member 16 by a detachable joint 30. Elements having reference numerals similar to previously utilized reference numerals are the same or similar, and operate in a manner that is the same or similar, as compared to the previously labeled elements.

FIG. 7C is a side view of the device of 7B, but further comprising SMP material actuator 60 that operates as described above in relation to previous embodiments. In accordance with embodiments of the present invention, a resilient member similar to actuating mechanism 14 described above (FIGS. 1A and 1B) or actuating struts 62 described above (FIGS. 6A and 6B) could be substituted for actuator 60. Illustratively, actuator 60, or the other chosen incorporated actuating mechanism, can be utilized to convert device 64 between a delivery configuration, as is illustrated in FIG. 7C and a deployed configuration, which is illustrated in FIG. 7D (described below). As described above, actuation could be temperature-based or mechanical-based, depending on the incorporated actuation mechanism.

FIG. 7D is a partially broken away side view that illustrates deployment of device 64 within an aneurysm 50. Illustratively, treatment device 64 operates in a manner similar to the above-described embodiments of device 10. Referring to FIG. 7D, in accordance with one embodiment, an embolic agent or other treatment agent can be delivered, along, the path demonstrated by arrows 56, through the elongated delivery member, through joint 30 (a detachment point), through a conduit formed through a retracted device 64 and into aneurysm 50.

In accordance with another embodiment of the present invention, a temporary actuation mechanism is used to transfer a neck bridge between delivery and deployed configurations. For example, a temporary actuation mechanism is first operably engaged to the neck bridge to convert it into, or maintain it in, the delivery configuration. Then, the mechanism is withdrawn or otherwise maneuvered to convert the neck bridge to the deployed configuration. After the neck bridge has been converted to the deployed configuration, the actuation mechanism is released from the neck bridge and removed from the patient's body. In accordance with one embodiment, a securing device such as a clip is utilized to maintain the neck bridge in the deployed configuration after the actuation mechanism has been removed.

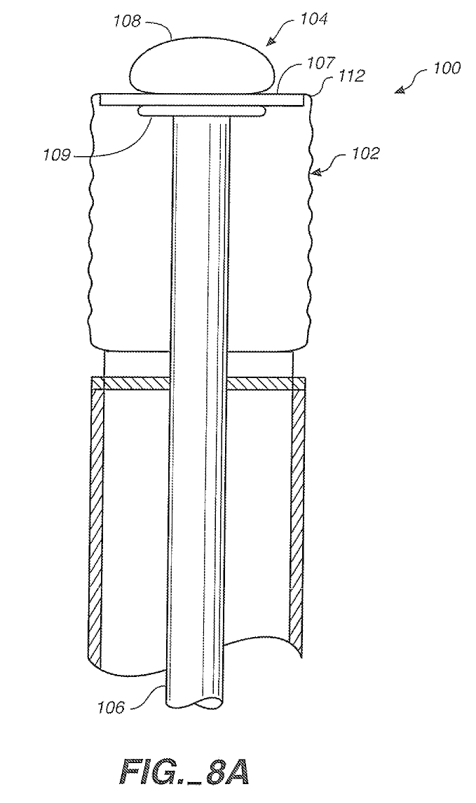

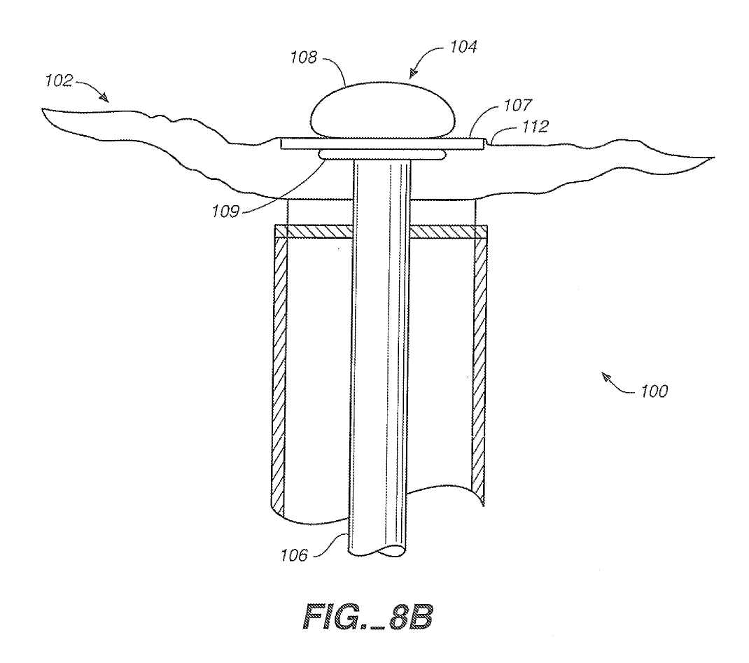

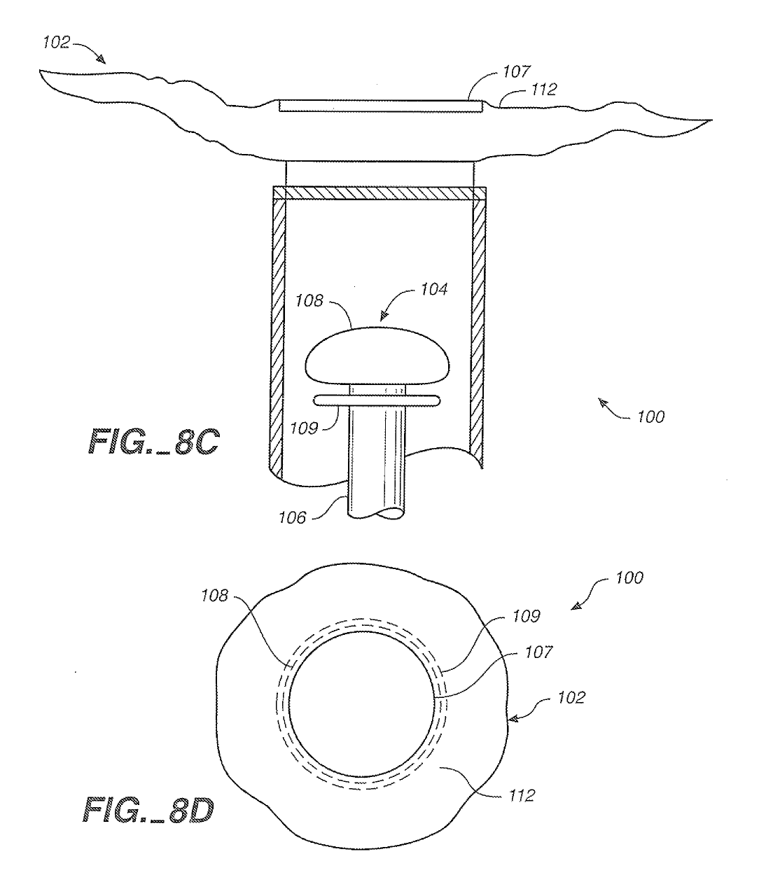

FIGS. 8A-8C are partially broken away side views of an aneurysm treatment device 100 in accordance with one embodiment of the present invention. Treatment device 100 operates in a manner similar to the above-described embodiments of devices 10 and 64, but device 100 incorporates a temporary actuation mechanism rather than a permanent one.

FIG. 8A shows treatment device 100 with a neck bridge 102 in a delivery configuration; FIG. 8B shows neck bridge 102 in a deployed configuration. A temporary actuation mechanism 104 is operably couplable to the collapsible neck bridge 102 and configured to facilitate conversion of bridge 102 between the delivery (8A) and deployed (8B) configurations. FIG. 8C shows actuation mechanism 104 being released from bridge 102 and removed from the patient's body.

Actuation mechanism 104 includes an elongated member 106 and a ball 108 attached or formed proximate a distal end of member 106. Elongated member 106 can be a guide wire, a catheter or some other similar elongated structure. Neck bridge 102 is illustratively guided (i.e., with guide wires etc.) to an internal treatment site within an aneurysm. Actuation mechanism 104 is advanced through neck bridge 102 until ball 108 is frictionally engaged by and then “pops” through a cylindrical opening formed in the distal end of bridge 102. Advancement and engagement can be done before or after bridge 102 is delivered to the treatment site. Rib 109, which illustratively has a diameter that is greater than the diameter of ball 108, internally engages the distal end of bridge 102 proximate the cylindrical opening. Rib 109 limits how far actuation mechanism 104 can be advanced through bridge 102. Applying pressure to the proximal end of mechanism 104 (i.e., by hand) enables pressure to be applied by rib 109 in order to maintain bridge 102 in the delivery configuration.

Ball 108 is adapted to again frictionally engage the cylindrical opening as actuation mechanism 104 is withdrawn from bridge 102, thereby converting neck bridge 102 to the deployed configuration. Following conversion, ball 108 “pops” through the cylindrical opening as mechanism 104 is withdrawn from the body.

FIG. 8D is a top view of device 100. The figure shows the cylindrical opening (identified as 106) formed in a distal end 112 of bridge 102. Cylindrical opening 106 can be reinforced with a rigid structure, such as a plastic ring. Ball 108 is shown inside of bridge 102 before it has been popped through opening 106. Rib 109 is shown inside of bridge 102 before it has internally engaged opening 106.

After ball 108 has been popped through opening 106, and after bridge 102 has been delivered to the treatment site, elongated member 106 is withdrawn such that ball 108 engages opening 106 and applies pressure to bridge 102, thereby transforming the bridge from the delivery configuration (FIG. 8A) to the deployed configuration (FIG. 8B). Once bridge 102 has achieved the deployed configuration, an additional amount of pressure can be applied to free ball 108 from neck bridge 102 by pulling ball 108 on through opening 106. Mechanism 104 can then be removed from the patient. Thus, actuation mechanism 104 is “temporary” in that it can be removed from the bridge, which can be left in its deployed and implanted state. In accordance with one embodiment, a clip or latch is utilized to maintain bridge 102 in the deployed configuration. Such clip or latch may be configured to automatically engage as bridge 102 is converted between the delivery and deployed configurations. In accordance with one embodiment, at least one of ball 108 and opening 106 includes a surface that is deformable to accommodate the described variable-pressure relationship between ball 108 and opening 106.

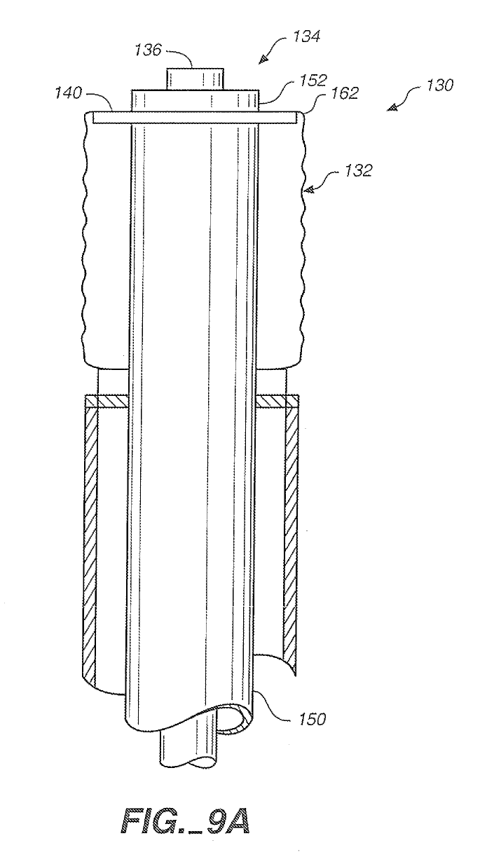

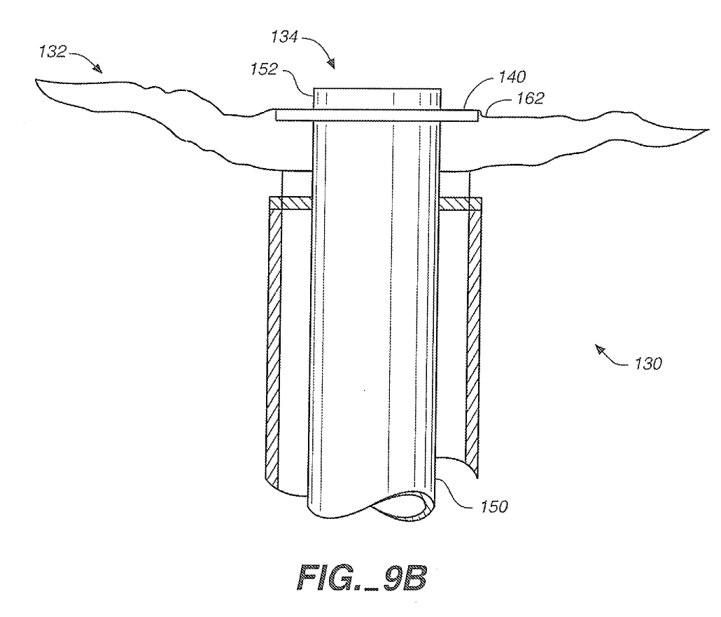

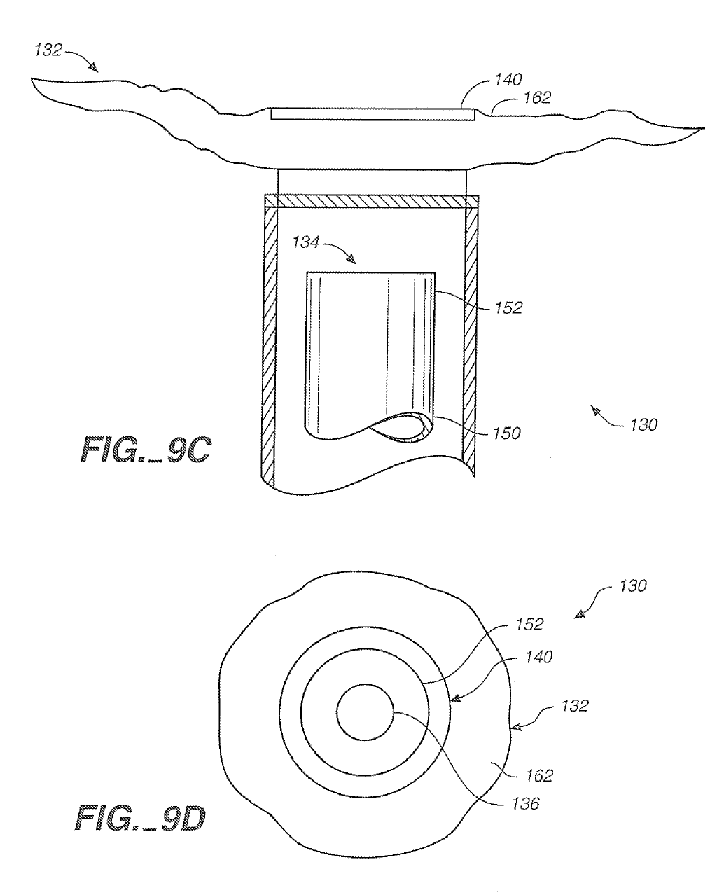

FIGS. 9A-9C are broken away side views of an aneurysm treatment device 130 in accordance with another embodiment of the present invention. Treatment device 130 operates similar to device 100 but incorporates a different embodiment of a temporary actuation mechanism.

FIG. 9A shows treatment device 130 with a neck bridge 132 in a delivery configuration while FIG. 9B shows device 130 with a neck bridge 132 in a deployed configuration. A temporary actuation mechanism 134 is operably couplable to the collapsible neck bridge 132 and configured to facilitate conversion of bridge 102 between the delivery (9A) and deployed (9B) configurations. FIG. 9C shows actuation mechanism 134 being released from bridge 132 and removed from the patient's body.

Actuation mechanism 134 includes an elongated member 150 having a tapered distal end 152. Elongated member 150 is illustratively, although not necessarily a catheter. Member 150 can be some other elongated device without departing from the scope of the present invention.

Actuation mechanism 134 is advanced through neck bridge 132 until tapered end 152 is frictionally engaged within a cylindrical opening formed in the distal end of bridge 132. This advancement and engagement can be done before or after bridge 132 is delivered to a treatment site within an aneurysm. End 152 is tapered such that its distal portion can slide through the cylindrical opening until a thicker portion prevents elongated member 150 from sliding further through the cylindrical opening. The increased thickness at the bottom of the taper effectively limits how far actuation mechanism 134 can be advanced through bridge 132.

Applying pressure to the proximal end of 134 (i.e., by hand) enables bridge 132 to be transferred to, or maintained in, the delivery configuration. Elongated member 150 is configured such that the thicker part of its tapered portion 152 continues to be frictionally engaged as actuation mechanism 134 is withdrawn from bridge 132, thereby converting neck bridge 132 to the deployed configuration. Following conversion, additional pressure is applied until the thicker part of tapered portion 152 pops loose from the cylindrical opening as mechanism 134 is removed from the body.

FIG. 9D is a top view of device 130. The Figure shows the cylindrical opening (identified as 140) formed in a distal end 162 of bridge 132. Cylindrical opening 140 can be reinforced with a rigid structure, such as a plastic ring. An optional guide wire 136 is shown in FIGS. 9A and 9D. Guide wire 136 is optionally utilized to assist in the delivery of bridge 132 to a treatment site, and/or to assist in guiding elongated member 150 through bridge 132. The tapered end 152 of elongated member 150 is shown in FIG. 9C before it has engaged opening 140.

After tapered end 152 has been engaged within opening 140, and after bridge 132 has been delivered to the treatment site, elongated member 150 is withdrawn such that pressure is applied to bridge 132, thereby transforming the bridge from the delivery configuration (FIG. 9A) to the deployed configuration (FIG. 9B). Once the bridge has achieved the deployed configuration, an additional amount of pressure can be applied to free the thick part of tapered portion 152 from opening 140 by pulling on the proximal end of elongated member 150. Mechanism 134 can then be removed from the patient. Thus, the actuation mechanism is temporary. A clip or latch can be utilized as described above to maintain the deployed configuration.

In accordance with another aspect of the present invention, neck bridge 102 and/or neck bridge 132 is detachably connected to an elongated delivery member that is independent of the actuation mechanism. The elongated delivery member is utilized to deliver the neck bridge to a treatment site. The actuation mechanism is maneuvered with (i.e., maneuvered around or through) the delivery member to affect actuation from the delivery to the deployed configuration. The elongated delivery member and the actuation mechanism are then removed from the patient leaving the neck bridge implanted at the treatment site. In accordance with one embodiment, the elongated delivery member is electrolytically detachable from the actuation mechanism similar to other embodiments described above.

In accordance with another aspect of the present invention, a hollow interior lumen is formed within actuation mechanism 104 and/or actuation mechanism 134. A treatment agent is illustratively delivered through the hollow interior lumen following conversion to the deployed configuration but prior to disengagement from the associated neck bridge.

Although the present invention has been described with reference to preferred embodiments, workers skilled in the art will recognize that changes may be made in form and detail without departing from the spirit and scope of the invention.

What is claimed is:

1. An aneurysm treatment device for at least partially closing a portion of a vascular aneurysm, comprising:
a neck bridge having a delivery configuration and a deployed configuration;
an actuation mechanism configured to be temporarily engaged to the neck bridge to convert the neck bridge between the delivery configuration and the deployed configuration.
